(19) United States
(12) Patent Application Publication
(10) Pub. No.: US 2011/0008804 A1
(43) Pub. Date: Jan. 13, 2011

(54) ANGIOPOIETIN-1 AND -2 BIOMARKERS FOR INFECTIOUS DISEASES THAT COMPROMISE ENDOTHELIAL INTEGRITY

(76) Inventors: Kevin C. Kain, Toronto (CA); W. Conrad Liles, Toronto (CA); Fiona E. Lovegrove, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/741,442

(22) PCT Filed: Nov. 5, 2008
(86) PCT No.: PCT/CA2008/001939

§ 371 Date: Sep. 22, 2010

Related U.S. Application Data
(60) Provisional application No. 60/985,508, filed on Nov. 5, 2007.

(51) Int. Cl. G01N 33/53

(52) U.S. Cl. 435/7.92

(57) ABSTRACT

The invention relates to a method of identifying a subject having, or at risk of developing, an infectious disease state wherein endothelial integrity is compromised comprising: (a) determining a test ANG-1 level in a sample from a subject; and (b) comparing the test ANG-1 level to a control level wherein lower test ANG-1 level compared to the control level is indicative of the subject developing said infectious disease state.

### FIELD OF THE INVENTION

The present invention relates to methods of identifying subjects having, or at risk of developing, infectious disease states that compromise endothelial integrity such as cerebral malaria, severe sepsis or hemorrhagic fevers.

### BACKGROUND OF THE INVENTION

Infectious diseases are an enormous health burden on the world's population. While some infectious diseases are relatively easy to diagnose and treat, others can progress rapidly to more complicated or severe forms or states that require serious attention and may prove fatal. For many infectious diseases, endothelial cell activation and dysfunction play a role in the pathogenesis of the disease.

Angiopoietins are glycoproteins that are involved in vascular development and angiogenesis. They regulate the integrity of the interface between neighbouring endothelial cells as well as the interface with surrounding matrix and mesenchyme. Four angiopoietins are known, named Angiopoietin-1 to Angiopoietin-4. Angiopoietin-1 (ANG-1) is a constitutively expressed molecule in many tissues that is responsible for maintaining vascular quiescence in the adult endothelium (21). The ANG-1 stabilizing effect is antagonized by angiopoietin-2 (ANG-2), which is released in response to stimuli such as injury, hypoxia and bacterial infection, and primes the endothelial activation response, promoting vascular permeability (21, 22). Elevated ANG-2 levels have been described in patients with severe compared to mild sepsis and may contribute to vascular leak in this disease process (23) Normally, ANG-1 levels are high and ANG-2 levels are low in the adult endothelium. An upregulation of ANG-2, or a dysregulation of the ANG-1/2 balance, may therefore be associated with disease states that compromise or disrupt endothelial integrity leading to vascular permeability or leakage.

Malaria remains the most important parasitic disease globally and is responsible for an estimated 500 million cases annually (1). Severe malarial complications occur primarily in Plasmodium falciparum infections and account for enormous morbidity and mortality in endemic regions. One of the more severe forms of malaria is cerebral malaria (CM), an encephalopathy associated with deep coma and a 15-40% mortality rate (2-4). Unfortunately, there are limited diagnostic tools available to determine which patients infected with P. falciparum will go on to develop cerebral complications. Furthermore, even in patients felt to have CM, the diagnosis is challenging. One third of those clinically diagnosed with CM will subsequently be shown to have alternative causes for their neurological syndrome (5).

Certain clinical symptoms are relatively sensitive predictive markers of fatal outcome in CM patients; however, people displaying these symptoms often die within 24 hours of admission to hospital due to the severity of their symptoms (6, 7, 9).

A number of studies have examined the correlation of peripheral biomarkers, such as cytokines, with severe disease states in malaria infection. Elevated levels of the pro-inflammatory cytokine tumour necrosis factor-alpha (TNF-α) have been associated with severe and cerebral malaria in a number of studies (10-15) and were identified as a strong predictor of mortality in CM (11, 13). However, other work has argued that there is no correlation between peripheral TNF-α levels and CM severity (16), and high serum TNF-α levels are often found in P. vivax malaria which does not cause CM (12).

Endothelial activation markers, such as the soluble cell-adhesion molecules, intercellular adhesion molecule-1 (sICAM-1), vascular cell adhesion molecule-1 (sVCAM-1) and endothelial leukocyte adhesion molecule-1 (sELAM-1) are upregulated in malarial infection and are positively correlated with disease severity (15, 18, 19). Additionally, acute endothelial activation, as measured by von Willebrand factor propeptide, is raised in severe malaria and drops rapidly following recovery from CM (20). Endothelial activation and endothelial cell dysfunction have also been suggested to play a role in the development of sepsis (30, 31), while new insights have also spurred interest in the possible role of angiogenic factors in the inflammatory response (32).

Previous work has shown correlations between the cytokine TNF-α (10-15) or soluble endothelial cell markers (15, 18, 19) and disease severity, but none of these studies have examined how robust these markers are to predict disease progression or outcome. Furthermore, clinically informative biomarkers with increased sensitivity and specificity (i.e., diagnostic accuracy) are needed in order to guide accurate and informed decision by health care providers and effective, targeted allocation of potentially costly health care resources. Well-validated biomarkers do not currently exist to accurately identify subjects at risk of developing infectious disease wherein endothelial cell integrity is compromised.

### SUMMARY OF THE INVENTION

The applicants disclose that Angiopoietin-1 (ANG-1), Angiopoietin-2 (ANG-2) and the ratio of ANG-2 to ANG-1 are biomarkers for subjects at risk of developing an infectious disease state wherein endothelial cell integrity is compromised. ANG-1 and ANG-2 levels were examined in serum samples from healthy controls and P. falciparum-infected patients with uncomplicated or cerebral disease. ANG-1 and ANG-2 levels were compared to TNF-α and soluble intercellular adhesion molecule 1 (ICAM), which have both been previously shown to correlate with severe or complicated malaria. ANG-1 levels were found to be significantly decreased and ANG-2 levels significantly increased in populations with cerebral versus uncomplicated malaria. The level of ANG-1 and the ratio of ANG-2:ANG-1 were found to be highly sensitive tests to identify CM. Receiver Operating Characteristic (ROC) curves were used to compare how well each test identifies cerebral compared to uncomplicated malaria patients and to determine a cut-off point between the populations. Furthermore, ANG-1 and ANG-2 levels were also found to correlate with the progression of symptoms of multiple organ failure in patients with severe sepsis.

Accordingly, one embodiment of the invention includes methods of identifying a subject having, or at risk of developing, an infectious disease state wherein endothelial cell integrity is compromised comprising:
(a) determining a test ANG-1 level in a sample from a subject; and
(b) comparing the test ANG-1 level to a control level wherein a lower test ANG-1 level compared to the control level is indicative of the subject having or developing said infectious disease state.

In another embodiment, the invention includes methods of identifying a subject having, or at risk of developing, an infectious disease state wherein endothelial cell integrity is disrupted comprising:
(a) determining a test ANG-1 level in a sample from a subject; and
(b) comparing the test ANG-1 level to a control level wherein a lower test ANG-1 level compared to the control level is indicative of the subject having or developing said infectious disease state.

In another embodiment, the invention comprises determining a test ANG-2 level in a subject, and comparing the test ANG-2 level to a control level, wherein increased test ANG-2 level is indicative of the subject having or developing said infectious disease state.

In another embodiment, the invention further comprises determining the ratio of test ANG-2 level to test ANG-1 level, where an increase in the ratio of test ANG-2/test ANG-1 compared to a control ratio or a decrease in the ratio of test ANG-1/test ANG-2 compared to a control ratio is indicative of the subject having or developing said infectious disease state.

In some embodiments of the invention, the infectious disease state comprises severe malaria or Cerebral Malaria (CM). In some embodiments the infectious disease state comprises sepsis, severe sepsis or septic shock. In other embodiments, the infectious disease state comprises dengue hemorrhagic fever or dengue shock syndrome.

In a further embodiment, the infectious disease state comprises a viral hemorrhagic fever. In some embodiments, the viral hemorrhagic fever is caused by the Marburg virus, Ebola virus or a rickettsial infectious agent.

In still further embodiments, the identity of the infectious disease is not known.

In one embodiment, the test sample comprises serum. In some embodiments, the subject is a human.

In some embodiments, more than one test ANG-1 level and/or ANG-2 level are determined at different time points to monitor the progression of an infectious disease state in said subject. In one embodiment, the methods described herein are used to monitor the progression of uncomplicated malaria to severe malaria or cerebral malaria. In another embodiment, the methods described herein are used to monitor the progression of an infection to sepsis, severe sepsis or septic shock.

The invention also includes a kit for determining whether a subject has, or is at risk for developing an infectious disease state wherein endothelial cell integrity is compromised, comprising an antibody directed against ANG-1 and/or an antibody directed against ANG-2. In some embodiments, the antibodies in the kit are detectably labeled. In a further embodiment, the kit comprises a medium suitable for formation of an antigen-antibody complex, and reagents for detection of the antigen-antibody complexes and instructions for the use thereof.

In one embodiment of the invention, the infectious disease state comprises an infectious disease state caused by exposure to a biowarfare agent. In a further embodiment of the invention, the biowarfare agent is selected from the group consisting of: anthrax, Ebola, Marburg virus, and the etiological agents of bubonic plague, cholera, tularemia, brucellosis, Q fever, machupo, coccidioidomycosis, glanders, melioidosis, shigellosis, Rocky Mountain spotted fever, typhus, psittacosis, yellow fever, Japanese B encephalitis, Rift Valley fever, and smallpox.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1: Comparison of Angiopoietins-1 and -2 with current markers of cerebral malaria in the Thai study population. Serum levels of angiopoietin-1 (A), angiopoietin-2 (B), the ratio of angiopoietin-2 to -1 (C), Tumour Necrosis Factor-alpha (TNFa, D), and soluble ICAM (E) were measured in 25 cerebral malaria (CM) patients, 25 uncomplicated malaria (UM) patients, and 10 healthy controls (HC).

FIG. 2: Receiver Operating Characteristic (ROC) curves for markers of cerebral malaria compared to uncomplicated malaria in the Thai study population. The ROC curve for each test is shown, with the null hypothesis as the diagonal for angiopoietin-1 (A), angiopoietin-2 (B), the ratio of angiopoietin-2 to -1 (C), Tumour Necrosis Factor-alpha (TNF-α, D), and soluble ICAM (E). The ROC curves for (A) and (C) are both lines at sensitivity equal to 1.00.

FIG. 3: Comparison of Angiopoietins-1, -2 and TNF-α as markers of cerebral malaria in the Ugandan study population. Serum levels of angiopoietin-1 (A), angiopoietin-2 (B), the ratio of angiopoietin 2 to 1 expressed as log base 10 (C), and tumour necrosis factor-alpha (TNF) (D) were measured in 28 healthy controls (HC), 67 uncomplicated malaria (UM) patients, and in 69 cerebral malaria (CM) patients.

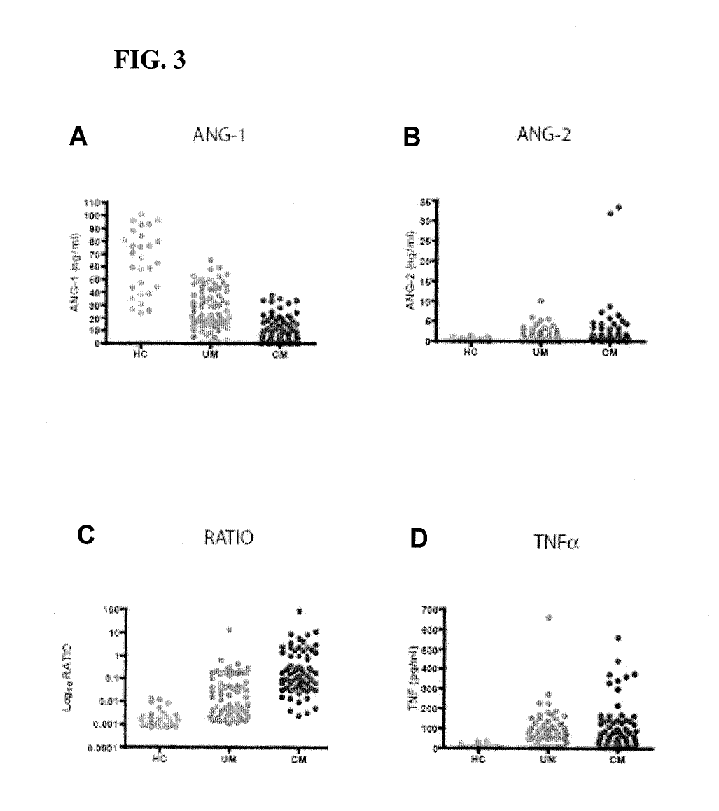

FIG. 4: Receiver Operating Characteristic (ROC) curves for markers of cerebral malaria compared to uncomplicated malaria in the Ugandan study population. The ROC curve for each test is shown, with the null hypothesis as the diagonal. Angiopoietin-1 (A), angiopoietin-2 (B), the ratio of angiopoietin-2 to -1 (C), and Tumour Necrosis Factor-alpha (TNF-α, D).

FIG. 5: Angiopoietin-1 levels are associated with clinical outcome in pediatric cerebral malaria patients from Uganda. Serum concentrations of angiopoietin-1 (ANG-1) were measured in 69 cerebral malaria (CM) patients at presentation and compared to outcome. Higher ANG-1 levels at presentation were associated with protection from fatal cerebral malaria. *p=0.027, non-fatal CM versus fatal CM (Wilcoxon rank-sum test).

FIG. 6: Angiopoietin-1 and -2 levels are correlated with next day MODS scores in patients with severe sepsis. Levels of ANG-2 (p<0.0001, FIG. 6A), ANG-1 (p=0.012, FIG. 6B) and the ratio of ANG-2/ANG-1 (p<0.0001, FIG. 6C) on day X were significantly correlated with MODS scores on day X+1.

FIG. 7: Angiopoietin-1 and -2 levels as predictors of mortality in severe sepsis. Mean ANG-2 levels were not significantly different between patients with severe sepsis who lived or died (p=0.059) (FIG. 7A), while peak ANG-1 levels were significantly different between patients with severe sepsis who lived or died (p=0.027) (FIG. 7B).

### DETAILED DESCRIPTION OF THE INVENTION

A desirable biomarker for infectious diseases would identify patients who are at increased risk of developing a severe disease state, so that these individuals may be more closely monitored and aggressively treated. The applicants disclose that ANG-1, ANG-2 and the ratio of ANG-1 to ANG-2 are useful biomarkers for identifying subjects having or at risk of developing an infectious disease state wherein endothelial cell integrity is compromised. The applicants have identified that ANG-1 and ANG-2, the normal balance of which maintains vascular endothelial integrity in its physiologically quiescent state, are dysregulated in cases of cerebral malaria. Furthermore, the applicants show that ANG-1 and the ratio of ANG-2 to ANG-1 are very specific and sensitive measures to identify patients affected by CM, and are useful biomarkers of cerebral malaria. The applicants have also shown that ANG-1 and ANG-2 levels can be used to predict the severity of symptoms in patients with severe sepsis and that peak ANG-1 levels can be used to predict mortality in patients with severe sepsis.

Accordingly, the present invention includes methods of identifying a subject having, or at risk of developing, an infectious disease state wherein endothelial cell integrity is compromised comprising:
(a) determining a test ANG-1 level in a sample from a subject; and
(b) comparing the test ANG-1 level to a control level wherein a lower test ANG-1 level compared to the control level is indicative of the subject having or developing said infectious disease state.

In a further embodiment, the invention provides for methods of identifying a subject having, or at risk of developing, an infectious disease state wherein endothelial cell integrity is compromised comprising:
(a) determining a test ANG-2 level in a sample from a subject; and
(b) comparing the test ANG-2 level to a control level wherein a higher test ANG-2 level compared to the control level is indicative of the subject having or developing said infectious disease state.

In one embodiment, the infectious disease state wherein endothelial cell integrity is compromised is cerebral malaria. In another embodiment, the infectious disease state wherein endothelial cell integrity is compromised is sepsis, severe sepsis or septic shock. In another embodiment, the infectious disease state is Dengue Hemorrhagic Fever or Dengue Shock Syndrome.

As used herein, “cerebral malaria” refers to a neurological condition associated with malaria infection. Optionally, the neurological condition includes, but is not limited to, coma or seizures. As used herein, “severe” or “complicated malaria” refers to subjects with malaria infection with signs of organ dysfunction. Optionally, signs of organ dysfunction include, but are not limited to, respiratory distress, acute renal failure or hypotension. As used herein, “uncomplicated malaria” refers to subjects with a malaria infection and fever, but without the presence of the symptoms of severe malaria or cerebral malaria. Malaria infection is caused by members of the plasmodium species. In one embodiment, the malaria infection is caused by P. falciparum, or P. vivax. A person skilled in the art will appreciate that malaria infection in a subject can be identified by methods known in the art, such as by positive identification of Plasmodium in a blood smear.

As used herein, “sepsis” refers to an infectious disease state characterized by a systemic inflammatory response causing endothelial dysfunction. Optionally, “severe sepsis” refers to sepsis further comprising organ failure or acute organ dysfunction. As used herein, “septic shock” refers to severe sepsis with refractory hypotension. In one embodiment, sepsis, is caused by a bacterial, viral, fungal, or parasitic infections. Optionally, sepsis is caused by infection with Staphylococcus aureus (including MRSA), Streptococcus pyogenes, Streptococcus pneumoniae, Pseudomonas aeruginosa, or Klebsiella pneumoniae

Optionally, signs of compromised endothelial integrity include peripheral or pulmonary edema, hypotension, shock and vascular leakage, and increased endothelial permeability or disruption of the endothelium.

Optionally, signs of severe sepsis include presence of infection, hyper- or hypothermia, elevated or depressed leukocyte count [WBC], hypotension, tachycardia, shock, acute respiratory failure [tachypnea, hypoxemia, hypercarbia], acute renal failure, acute hepatic dysfunction, and neurologic dysfunction. Optionally, the severity of organ dysfunction in severe sepsis may be categorized using MODS (Multiple Organ Dysfunction Score) scores as described (28).

Optionally, in one embodiment, clinical signs of a subject having compromised endothelial integrity include one or more of the following: >20% rise in haematocrit controlled for age and sex of the subject, >20% drop in haematocrit following treatment with fluids as compared to baseline, signs of plasma leakage such as pleural effusion, ascites of hypoproteinaemia.

The term “identifying” as used herein refers to a process of determining a subject's likelihood of having, or risk of developing an infectious disease state wherein endothelial cell integrity is compromised. As used herein, identifying a subject at risk of developing an infectious disease state wherein endothelial cell integrity is compromised includes identifying a subject at risk of progressing to a more severe form of the disease state. Accordingly, the invention can be used to detect or monitor the appearance and progression of infectious disease in a subject. In one embodiment, the methods are used to provide a prognosis for a subject with an infectious disease. The applicants note that it is not necessary to know the identity of the specific causative infectious agent in order to determine a subject's risk for developing said infectious disease state.

The term “subject” as used herein refers to any member of the animal kingdom. In one embodiment the subject is a mammal, such as a human.

The term “sample” refers to any fluid or other specimen from a subject which can be assayed for ANG-1 or ANG-2 protein levels, for example, blood, serum or plasma. In one embodiment, levels of ANG-1 and/or ANG-2 are determined in a test sample from a subject.

The terms “ANG-1 level”, “ANG-2 level” or “control level” refer to the relative or absolute amount of the relevant protein in the sample.

The term “control level” refers to a level of the ANG-1 or ANG-2 protein in a sample from a subject or group of subjects that are infected with an infectious disease but do not develop a severe or complicated infectious disease state wherein endothelial cell integrity is compromised. In one embodiment, the infectious disease is uncomplicated malaria and said infectious disease state wherein endothelial cell integrity is compromised is severe malaria or cerebral malaria. In another embodiment, the infectious disease is dengue fever, and the infectious disease state wherein endothelial cell integrity is compromised is Dengue Hemorrhagic Fever or Dengue Shock Syndrome. In a further embodiment, the “control level” is determined from a subject or group of subjects known to have an infectious disease state that does not progress to a severe infectious disease state characterized by disruption of endothelial integrity or vascular leakage. In one embodiment the infectious disease state is sepsis, severe sepsis or septic shock and the control levels are taken subjects with uncomplicated infections. In a further embodiment the controls are age-matched controls.

The term “control level” optionally includes levels of ANG-1 and/or ANG-2 protein determined from healthy subjects that are not suffering from infectious disease. The term also includes pre-determined standardized results. Optionally, the term “control level” includes levels of ANG-1 and/or ANG-2 protein from a test sample from a subject determined at an earlier time point. A person skilled in the art will appreciate that a “control ratio” may easily be derived from the ratio of two “control levels”.

In a further embodiment, the method includes comparing levels of ANG-1 and/or ANG-2 in samples taken from a subject at different time points. Accordingly, the methods described herein may be used to monitor the progression of an infectious disease state in a subject or group of subjects at different time points. In one embodiment, a test sample is taken from a subject and subsequent samples are taken at periodic intervals of between 1 hour and 14 days. In one embodiment, test samples are taken at periodic intervals of approximately 1 hour, 2 hours, 4 hours, 8 hours, 12 hours, 24 hours or 48 hours.

As shown in Example 7, in subjects with severe sepsis ANG-1 and/or ANG-2 levels are correlated with the progression of the disease to more serious disease states as indicated by higher MODS scores. Accordingly in one embodiment ANG-1 and/or ANG-2 levels are used to identify subjects at risk of developing sepsis, severe sepsis or septic shock. In a further embodiment, ANG-1 and/or ANG-2 levels are monitored at different time points in a subject and used to follow the progression of the disease state. In yet another embodiment, peak ANG-1 levels are used to predict the risk of developing a more severe disease state characterized by organ dysfunction, disruptions in endothelial cell integrity or vascular leakage. In one embodiment, peak ANG-1 levels are used to predict mortality in a subject or subjects with severe sepsis.

Severe infectious diseases wherein endothelial cell integrity is compromised have a high mortality rate. It is therefore important to have a highly sensitive test—one that predicts with high accuracy who is at risk of developing severe disease. As described in Example 1 and shown in Table 4, a decrease in serum ANG-1 levels below the threshold of 20 (i.e., 21.26) ng/ml or increase in ANG-2:ANG-1 ratio above 0.1 (i.e., 0.131) ng/ml accurately identified 100% of the CM cases in the Thai study population. A normal range or control level is readily determined for other at-risk populations. Using the ANG-1 serum levels and ANG-2:ANG-1 ratio as biomarkers of CM is superior to the previously identified markers TNF-α and ICAM-1. A person skilled in art may readily set suitable thresholds or cut-off points for additional populations where there are different levels of ANG-1 and/or ANG-2 protein in response to cerebral malaria or other severe infectious disease states. In one embodiment, the cut-off points for identifying CM are between 15 and 22 ng of ANG-1 protein per ml of serum.

In one embodiment, the magnitude of a subjects risk for developing a severe infectious disease state wherein endothelial cell integrity is compromised is indicated by comparing the level of ANG-1 or ANG-2 in a subject to a control level. In a further embodiment, the ratio of ANG-2:ANG-1 ratio is useful to identify subjects at risk of developing infectious disease wherein endothelial cell integrity is compromised and also to serve as an indication of the severity of that risk. It is an embodiment of the invention that the identification of a subject at risk of developing a severe infectious disease state according to the current invention will provide useful information for health care resource allocation and disease prognosis. In one embodiment, the methods described by the inventors provide information on the risk of a subject or group of subjects developing severe disease wherein endothelial cell integrity is compromised in response to biowarfare agents.

The applicants disclose that ANG-1 and ANG-2 and the ANG-2 to ANG-1 ratio are useful biomarkers of endothelial dysfunction in severe infectious disease and are predictive of severe infectious and inflammatory disease states wherein endothelial cell integrity is compromised. Examples of such severe disease states include dengue shock syndrome/dengue hemorrhagic fever; viral hemorrhagic fevers, and rickettsial infections that affect the vasculature and vascular permeability. Dengue infection causes a non-specific febrile illness, which may progress to hemorrhagic fever during the later stages (secondary) stage of illness. One of the more commonly detectable symptoms of this disease progression is vascular leak (25). ANG-1 is a key modulator of vascular integrity, and the present invention measures downregulation of ANG-1 either in response to, or as a result of, dengue hemorrhagic fever. Viral hemorrhagic fevers, including those induced by the Marburg and Ebola viruses (26), are also readily monitored with the present invention by detecting changes in ANG-1 or ANG-2 or an ANG-1/ANG-2 imbalance.

The applicants have also provided data showing that ANG-1 and the ANG-2:ANG-1 ratio are superior biomarkers to those presently accepted for malaria. In a further embodiment of the invention, the detection of ANG-1 and/or ANG-2 biomarkers provides information useful for diagnosing or prognosing an infectious disease state in a subject. In another embodiment, the detection of ANG-1 and/or ANG2 biomarkers provides information useful for diagnosing or prognosing cerebral malaria or severe malaria. The applicants have also provided data showing that ANG-2 and ANG-1 levels are useful for diagnosing or prognosing sepsis, severe sepsis or septic shock. In a further embodiment, the detection of ANG-1 and/or ANG-2 biomarkers provides information useful for diagnosing or prognosing dengue fever, dengue hemorrhagic fever or dengue shock syndrome.

A person skilled in the art will appreciate that a number of different methods are useful to determine the level of the relevant proteins of the invention. In one embodiment, protocols for determining the level of protein use agents that bind to the protein of interest, namely ANG-1 or ANG-2. In one embodiment the agents are antibodies or antibody fragments.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies having specificity for ANG-1 or ANG-2 may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

In one embodiment of the invention, the agents, such as antibodies or antibody fragments, that bind to the proteins of interest, ANG-1 and/or ANG2, are labeled with a detectable marker.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I or ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a labeled secondary antibody can be used to detect the protein of interest.

A person skilled in the art will appreciate that a number of other methods are useful to determine the levels of ANG-1 and or ANG-2 protein in a sample, including immunoassays such as Western blots, ELISA, and immunoprecipitation followed by SDS-PAGE immunocytochemistry. In addition, protein arrays (including microarrays) are useful.

Furthermore, in one embodiment of the invention, additional clinically relevant biomarkers are tested along with ANG-1 and/or ANG-2, such as specific pathogen-associated antigens.

Any of the described methods of the invention to provide a prognosis for severe infectious disease wherein endothelial cell integrity is compromised are useful in addition or in combination with diagnostic techniques for infectious disease known in the art.

In one embodiment of the invention, ANG-1 and/or ANG-2 protein levels and any additional markers of interest are determined using multiplex technology. This technology has the advantage of quantifying multiple proteins simultaneously in one sample. The advantages of this method include low sample volume, cost effectiveness and high throughput screening. Antibody-based multiplex kits are available from Linco (Millipore Corporation, MA), Bio-Rad Laboratories (Hercules, Calif.), Biosource (Montreal, Canada), and R&D Systems (Minneapolis, Minn.).

The invention also includes kits for identifying subjects at risk of developing an infectious disease state wherein endothelial cell integrity is compromised comprising a detection agent for ANG-1 and/or ANG-2, typically with instructions for the use thereof. In a further embodiment, the kit includes antibodies directed against ANG-1 and/or ANG-2, optionally with one or more of a medium suitable for formation of an antigen-antibody complex, reagents for detection of the antigen-antibody complexes and instructions for the use thereof. In an additional embodiment, the invention relates to a composition comprising an anti-ANG-1 antibody and an anti-ANG-2 antibody, optionally provided together in a container.

The present invention also has the desirable characteristic of providing an indication of the risk of a subject developing an infectious disease state wherein endothelial cell integrity is compromised without requiring the identification of the specific cause of disease or infectious disease agent.

The methods of this invention are useful to determine whether a subject, such as a health care worker or soldier, has been exposed to an infectious disease or infectious disease agent that results in compromised endothelial cell integrity, by measuring ANG-1 and/or ANG-2 levels as described herein. Similarly, where it is known that a subject has been exposed, the measurements are useful to determine whether the subject is at risk of developing severe complications due to exposure to the disease or agent. Decisions regarding patient care or level of intervention required during an outbreak or suspected outbreak of infectious disease can reflect the information provided by the methods of the current invention.

For example, the methods of the current invention could be used to provide information regarding risk when faced with exposure to biowarfare agents such as anthrax, Ebola virus, Marburg virus, or the causative microbial agents of bubonic plague, cholera, tularemia, brucellosis, Q fever, machupo, coccidioidomycosis, glanders, melioidosis, shigellosis, Rocky Mountain spotted fever, typhus, psittacosis, yellow fever, Japanese B encephalitis, Rift Valley fever and smallpox. It is an advantage of the current invention that the precise causative agent does not need to be known in order to identify subjects that are at risk of developing an infectious disease state wherein endothelial cell integrity is compromised.

The above disclosure generally describes the present invention. A more complete understanding can be obtained by reference to the following specific examples of certain embodiments of the invention.

### Example 1

### Angiopoietin-1 and -2 Levels in an Adult Thai Study Population and Pediatric Ugandan Study Population

Thai Study Population. Adults living in Thailand and undergoing treatment at the Hospital for Tropical Disease (Mahidol University) were recruited to this study. Blood samples were collected from 50 subjects with P. falciparum malaria (25 uncomplicated malaria and 25 cerebral malaria) and from 10 healthy control subjects, who had no malaria exposure as shown in Table 1. Uncomplicated malaria subjects were classified based on a positive blood smear for P. falciparum and fever, without the presence of severe malaria symptoms, as defined by the World Health Organization criteria (2), but not cerebral malaria. Cerebral malaria was defined as unrousable coma (Glasgow coma scale ≦8) in P. falciparum infection where other causes of coma were excluded (2).

Ugandan Study Population. Children 4-12 years old admitted to Mulago Hospital in Uganda were eligible for enrolment if they had uncomplicated malaria or met the WHO criteria for complicated malaria: P. falciparum on blood smear and coma (Blantyre coma scale ≦2 or Glasgow coma scale ≦8) not attributable to hypoglycemia, convulsions, meningitis or other identifiable cause (2). The Ugandan study population is presented in Table 1, and is also described in (27). Lumbar punctures were performed to rule out meningitis/encephalitis. Children were considered to have UM if they had fever (or a history of fever within 24 hours), P. falciparum infection on blood smear, but no evidence of severe or complicated malaria (2). Healthy controls were recruited from the extended household areas of children with cerebral or uncomplicated malaria and were determined to be healthy by medical history (with no malaria history for the previous 6 months), physical examination and microscopic examination of blood smears. The Ugandan study population (shown in Table 1), has been previously described (27).

### Measurement of Angiopoietin-1 and -2

The concentration of ANG-1 and ANG-2, TNF-α and sICAM-1 (Thai study sample only) were measured in serum samples using a standard sandwich enzyme-linked immunosorbant assay (ELISA) according to the manufacturer's instructions (ANG-1, -2, and sICAM-1: R&D Systems, Minneapolis Minn.; TNF-α: eBioscience, SanDiego Calif.). Serum samples were diluted 1:10-1:20 for ANG-1 and sICAM-1, 1:5 for ANG-2, and 1:2 for TNF-α in PBS/1% BSA, so that the sample values fell within the ELISA range of detection. Concentrations were interpolated from 4-parameter-fit standard curves generated using a standard curve of recombinant human proteins. TNF levels in Ugandan children were measured as described (27).

### Statistical Analysis

Statistical analysis was performed using GraphPad Prism™ (v4.03). Serum protein levels were analyzed using a Kruskal-Wallis test, followed by Dunn's multiple comparison tests. Receiver operating characteristic (ROC) curves and area under the ROC curves were generated using SPSS (v11). Cutoff values were derived mathematically from the ROC curves, using the point on the ROC curve with the lowest value for the formula: (1−sensitivity)²+(1−specificity)². Sensitivity and specificity were calculated using standard formulas. Angiopoietin levels and survival outcomes were analyzed using the Wilcoxon rank-sum test.

### Results

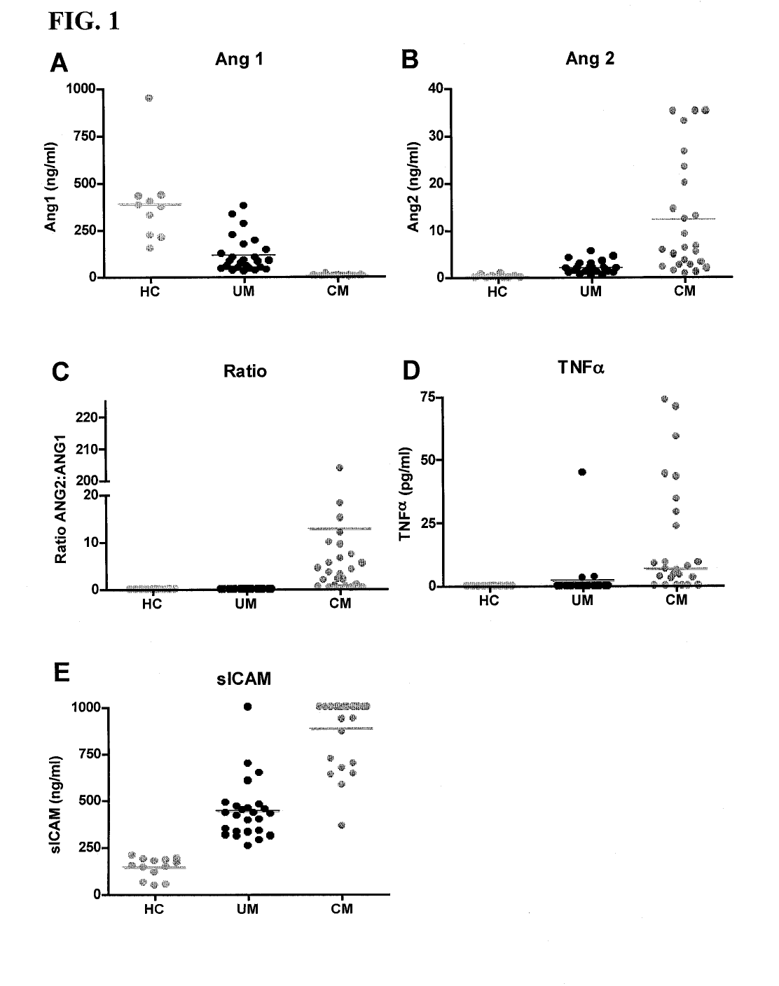

Thai Study Population. Angiopoietin protein levels were measured in the serum of the Thai study population of healthy controls (HC), uncomplicated malaria (UM) subjects and cerebral malaria (CM) subjects. The pro-quiescent ANG-1 was significantly decreased in CM compared to UM or HC, but not in UM compared to HC (FIG. 1A; Kruskal-Wallis: HC vs. CM p<0.001, UM vs. CM p<0.001). Additionally, the pro-inflammatory ANG-2 was significantly increased in CM compared to UM or HC, and also in UM compared to HC (FIG. 1B; Kruskal-Wallis: HC vs. UM p<0.01, HC vs. CM p<0.001, UM vs. CM p<0.01). As an additional measure, the ratio of ANG-2 to ANG-1 for each subject was found to be significantly different between HC, UM and CM (FIG. 10; Kruskal-Wallis: HC vs. UM p<0.05, HC vs. CM p<0.001, UM vs. CM p<0.001). To compare the novel markers ANG-1 and ANG-2 to known markers of cerebral malaria, TNF-α and ICAM-1 were also tested in the same sample set. TNF-α is significantly increased in CM compared to either HC or UM (FIG. 1D; Kruskal-Wallis: HC vs. CM p<0.001, UM vs. CM p<0.001), however overall levels of TNF-α, even in positive samples, were very low. Finally, sICAM-1 levels were significantly increased in CM versus HC or UM and in UM compared to HC (FIG. 1E; Kruskal-Wallis: HC vs. UM p<0.01, HC vs. CM p<0.001, UM vs. CM p<0.001). Additionally, the median and range for each group and marker tested are presented in Table 2. Of note, there is no overlap in the ranges of CM and UM patients in the ANG-1 and ANG-2: ANG-1 ratios, indicating that these markers clearly discriminated the respective groups, whereas some overlap occurs between patient groups in the ANG-2, sICAM-1 and TNF-α tests.

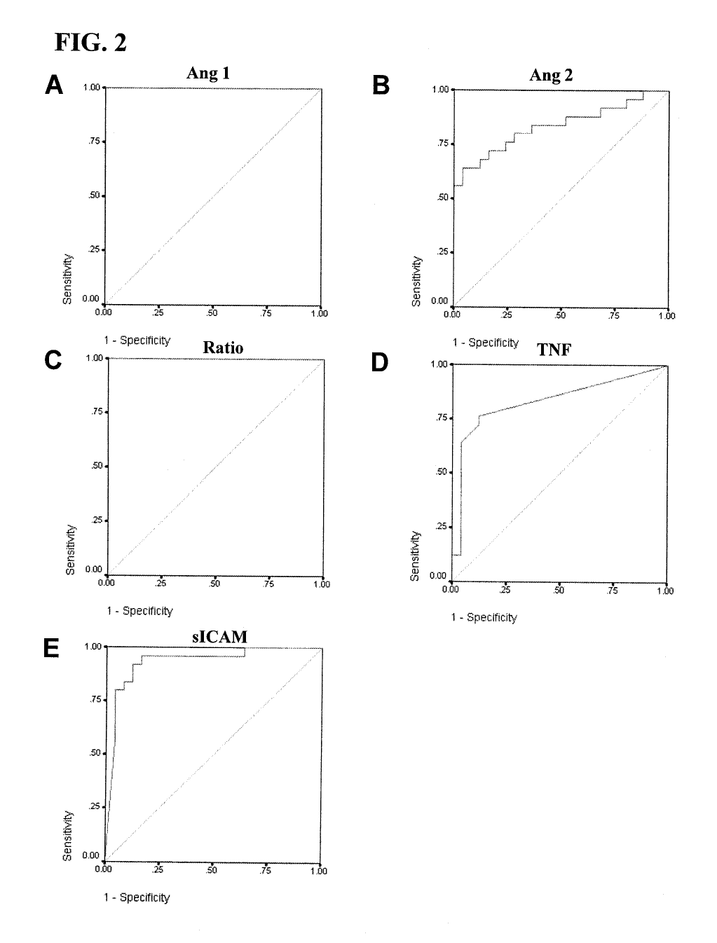

Ugandan Study Population. Angiopoietin levels were also examined in a larger cohort of Ugandan children. Similar to the observations in Thailand, serum ANG-1 levels were significantly decreased in Ugandan children with CM compared to Ugandan children with UM and healthy controls, and in Ugandan children with UM compared to healthy controls (FIG. 2A; Kruskal-Wallis: p<0.001). Additionally, ANG-2 levels were significantly elevated in children with CM compared to children with UM and healthy controls (FIG. 2B; Kruskal-Wallis: p<0.001), and between children with UM and healthy controls (p<0.01). Furthermore, as in the adult population, the ANG-2:ANG-1 ratio was significantly higher in children with CM than in children with UM and healthy controls, and in children with UM compared with healthy controls (FIG. 2C; Kruskal-Wallis: p<0.001). While TNF levels were significantly lower in healthy controls compared to children with UM and children with CM (FIG. 2D; Kruskal-Wallis: p<0.001), there was no significance difference in serum TNF values between children with CM and children with UM. The median and range for each group and marker tested for the Ugandan study population are presented in Table 2 along with the data from the Thai study population. There was some overlap in the concentration ranges in the Ugandan children with UM and the Ugandan children with CM.

TABLE 1
Demographic information for adult malaria patients from Thailand and
pediatric malaria patients from Uganda; healthy controls (HC), uncomplicated
malaria patients (UM) and cerebral malaria patients (CM). Age and parasitemia
are presented as median (range).

| | Adult (Thailand) | | | Pediatric (Uganda) | | |
|---|---|---|---|---|---|---|
| Group | N | Age | Parasites/ml | N | Age | Parasites/ml |
| HC | 10 | 32 (25-48) | 0 | 28 | 7 (3.2-12) | 0 |
| UM | 25 | 22 (14-63)* | 2.2 × 10⁴ | 67 | 7 (3-12) | 3.3 × 10⁴ |
| | | | (170-1.9 × 10⁵)* | | | (48-2.4 × 10⁵)* |
| CM | 25 | 25 (17-50) | 3.1 × 10⁵ | 69 | 5.4 (3.2-12)*† | 4.0 × 10⁵ |
| | | | (500-2.1 × 10⁶)*† | | | (32-9.3 × 10⁵)* |

*p < 0.05 vs. HC and
†p < 0.05 vs. UM (Kruskal-Wallis test with Dunn's multiple comparison post-test).

TABLE 2
Biomarker levels in serum of healthy controls (HC), uncomplicated malaria
patients (UM) and cerebral malaria patients (CM) from adult Thai patients and
pediatric Ugandan patients. Values are presented as median (range).

| | Adult (Thailand) | | | Pediatric (Uganda) | | |
|---|---|---|---|---|---|---|
| Marker | HC | UM | CM | HC | UM | CM |
| ANG-1 | 378 | 82.25 | 3.51 | 64.4 | 25.0 | 9.0 |
| (ng/ml) | (151-946) | (27.3-379) | (0.001-15.3) | (23.5-101) | (0.39-64.9) | (0.39-37.5) |
| ANG-2 | 0.0089 | 1.84 | 6.19 | 0.068 | 0.28 | 0.83 |
| (ng/ml) | (0.005-0.847) | (0.25-5.44) | (0.78-35) | (0.068-1.33) | (0.068-10.0) | (0.068-33.5) |
| Ratio | 0.00003 | 0.017 | 3.47 | 0.0015 | 0.013 | 0.14 |
| (ANG-2/ANG-1) | (0.000013-0.0021) | (0.03-0.11) | (0.15-204) | (0.00071-0.014) | (0.0011-13.0) | (0.0024-81.5) |
| TNF | 0 | 0 | 6.51 | 0 | 70.6 | 76 |
| (pg/ml) | (0-0) | (0-44.8) | (0-73.8) | (0-31.3) | (0-658) | (0-559) |
| sICAM | 151.0 | 426.5 | 1000 | n/a | n/a | n/a |
| | (46.98-206.8) | (259.4-1000) | (364.5-1000) | | | |

### Example 2

### Receiver Operating Characteristic (ROC) Curves Indicate that ANG-1 Perfectly Discriminates Between Uncomplicated and Cerebral Malaria Subjects, and Out-Performs Other Standard Biomarkers

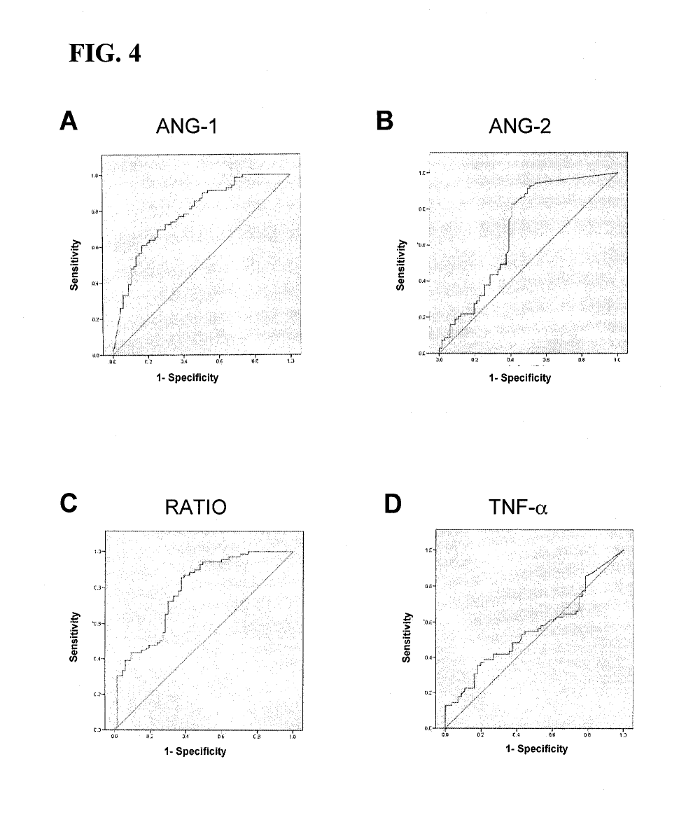

The ROC curve shows the ability of a test to discriminate between subjects with and without disease (24), and in this example, with or without cerebral complications in malaria infection. ROC curves for each biomarker, examining CM patients as “cases” and uncomplicated malaria patients as “controls”, were plotted and compared to assess the ability of each marker to discriminate between patients with and without cerebral complications (FIGS. 2 and 4, Table 3). In the Thai population, ANG-1 and the ANG-2:ANG-1 ratio have an area under the curve (AUC) of 1 (FIG. 2, Table 3) and differ significantly (p<0.001) from that of a chance result (AUC: 0.5). This finding was validated in the geographically, genetically and demographically distinct Ugandan pediatric population, where ANG-1 (AUC: 0.785, p<0.001) and the ANG-2:ANG-1 ratio (AUC: 0.779, p<0.001) were still the best of the biomarkers examined (FIG. 4, Table 3; sICAM-1, data not shown). Although ANG-2 did not have such large AUC values, it showed moderate accuracy as a discriminatory marker in both populations examined (FIG. 2—Thai: AUC=0.835, p<0.001; FIG. 4—Uganda: AUC=0.688, p<0.001).

TABLE 3
Area under ROC curve (AUC) for each test
comparing UM with CM patients.

| | Adult (Thailand) | | Pediatric (Uganda) | |
|---|---|---|---|---|
| Marker | AUC (95% Cl) | P | AUC (95% Cl) | P |
| ANG-1 | 1 (1-1) | <0.001 | 0.785 (0.709-0.861) | <0.001 |
| ANG-2 | 0.835 (0.719-0.951) | <0.001 | 0.688 (0.595-0.780) | <0.001 |
| Ratio | 1 (1-1) | <0.001 | 0.779 (0.702-0.856) | <0.001 |
| TNF | 0.834 (0.713-0.955) | <0.001 | 0.557 (0.453-0.661) | 0.268 |

P values are based on the null hypothesis that AUC = 0.5

Compared to ANG-1 and ANG-2 as biomarkers of CM, previously studied markers of severe and CM such as TNF had moderate accuracy as a discriminating test (FIG. 2D, Table 3, AUC: 0.834, p<0.001) in Thai adults; however, TNF was a poor discriminator between CM and uncomplicated malaria in the Ugandan pediatric population (FIG. 4D, Table 3, AUC: 0.557, p=0.268).

### Example 3

### ANG-1 Shows High Sensitivity and Specificity as a Biomarker of Cerebral Malaria, Based on a Predicted Cut-Off Value Derived from the ROC Curve

For each of the tests, a cut-off value to discriminate between CM cases and UM controls was derived from the ROC curve. The diagnostic accuracy (sensitivity, specificity, positive and negative likelihood ratios) for each biomarker, stratified by patient population, are reported in Table 4. Based on ROC curve analysis, ANG-1 best discriminated CM from UM. In the Thai population, ANG-1 at a threshold of 21 ng/mL had a sensitivity and specificity of 100% for distinguishing CM from UM, indicating that these tests correctly identified CM cases 100% of the time and equally correctly identified UM controls. sICAM-1 also showed a sensitivity of (0.92), with a specificity of 0.88. ANG-2 and TNF-α had similar specificities (0.84 and 0.88, respectively), although showed much lower sensitivity than the other tests (ANG-2: 0.72, TNF-α: 0.76).

For Ugandan children ANG-1 (at a cut off of 15 ng/mL) distinguished CM from UM the sensitivity and specificity of 70% and 75%, respectively. Across both populations, using an ANG-1 threshold of 15 ng/mL, the pooled sensitivity (95% CI) was 0.77 (0.67-0.84), specificity 0.82 (0.72-0.88), likelihood ratio of CM given a positive test (ANG-1 below 15 ng/mL) was 4.1 (2.7-6.5) and the likelihood ratio of CM given a negative test was 0.29 (0.20-0.42).

TABLE 4
Optimal cut-off values (95% CI) for each test and sensitivity (SEN),
specificity (SPEC), positive likelihood ratio (LR(+)) and negative likelihood ratio (LR(−))
at the chosen cut-off value comparing uncomplicated malaria with cerebral malaria patients.

| | Adult (Thailand) | | | | | Pediatric (Uganda) | | | | |
|---|---|---|---|---|---|---|---|---|---|---|
| | Cut- | | | | | Cut- | | | | |
| Marker | off | SEN | SPEC | LR(+) | LR(−) | off | SEN | SPEC | LR(+) | LR(−) |
| ANG-1 | 21.26 ng/ml | 1 | 1 | ∞* | 0* | 15.05 ng/ml | 0.70 | 0.75 | 2.7 | 0.40 |
| | | (0.87-1) | (0.87-1) | | | | (0.58-0.79) | (0.63-0.83) | (1.8-4.3)* | (0.28-0.60)* |
| ANG-2 | 3.04 ng/ml | 0.72 | 0.84 | 4.5 | 0.33 | 0.39 ng/ml | 0.83 | 0.60 | 2.1 | 0.29 |
| | | (0.52-0.86) | (0.65-0.94) | (1.8-11)* | (0.17-0.64)* | | (0.72-0.90) | (0.48-0.71) | (1.5-2.8)* | (0.17-0.51)* |
| Ratio | 0.131 | 1 | 1 | ∞* | 0* | 0.052 | 0.73 | 0.70 | 2.4 | 0.39 |
| | | (0.87-1) | (0.87-1) | | | | (0.61-0.82) | (0.58-0.79) | (1.6-3.6)* | (0.26-0.59)* |
| TNF | 1.46 pg/ml | 0.76 | 0.88 | 6.3 | 0.27 | 81.1 pg/ml | 0.48 | 0.62 | 1.3 | 0.82 |
| | | (0.57-0.89) | (0.70-0.96) | (2.1-19)* | (0.13-0.56)* | | (0.36-0.61) | (0.49-0.74) | (0.84-2.0) | (0.60-1.1) |

*significantly different from 1 (p < 0.05).

### Example 4

### The Association of ANG-1 with CM is Independent of Parasite Burden and Other Covariates

Although higher parasite burdens are associated with an increased risk of severe or CM, these complications can occur in individuals with relatively low peripheral parasitemias. In the Thai population, patients with CM had significantly higher parasitemias than in uncomplicated malaria patients (p<0.001); however, this was not the case in Ugandan children (Table 1). Increased serum cytokine levels may reflect the immune response to increased parasite burdens, rather than being indicative of a clinical syndrome such as CM. In support of this hypothesis, TNF levels were significantly correlated with the parasite burden among Ugandan children with UM (r²=0.38, p=0.004) and CM (r²=0.44, p<0.001). In contrast, angiopoietins did not significantly correlate with parasitemia in an analysis stratified by clinical syndrome and patient population, yet were strongly associated with CM, suggesting that they provide prognostic information independent of the parasite burden.

Multivariable logistic regression modeling was used to examine the independent predictive value of biomarkers on outcome (CM vs. UM) in order to account for potential confounding effects of multiple covariates (SPSS 16.0). Hosmer Lemeshow test was used to verify model goodness of fit. ANG-1 (but not TNF) was independently associated with CM in a multivariate logistic regression model, adjusting for the potential confounding effects of multiple covariates as shown in Table 5.

TABLE 5
Results of a multivariate logistic regression model to
predict CM (versus UM) in two diverse patient populations.

| Predictor | Adjusted OR (95% C1) | P value |
|---|---|---|
| Group: | | |
| Thailand | 1.0* | |
| Uganda | 0.36 (0.029-4.7) | 0.44 |
| Age | 0.96 (0.86-1.1) | 0.53 |
| Parasitemia | 1.00 (1.00-1.00) | 0.20 |
| (parasites/μL) | | |
| ANG-1 (ng/mL) | 0.899 (0.864-0.934)** | <0.001 |
| ANG-2 (ng/mL) | 1.10 (0.944-1.28) | 0.22 |
| Ratio | 1.01 (0.932-1.09) | 0.82 |
| (ANG-2/ANG-1) | | |
| TNF (pg/mL) | 1.00 (0.994-1.003) | 0.91 |

(*baseline comparator group)
(**Adjusted odds ratio represents the incremental odds of CM for every unit increase (1 ng/mL) in the ANG-1 level.)

### Example 5

### Angiopoietin-1 Levels and the Angiopoietin-2/Angiopoietin-1 Ratio Predict Survival in African Children with Cerebral Malaria

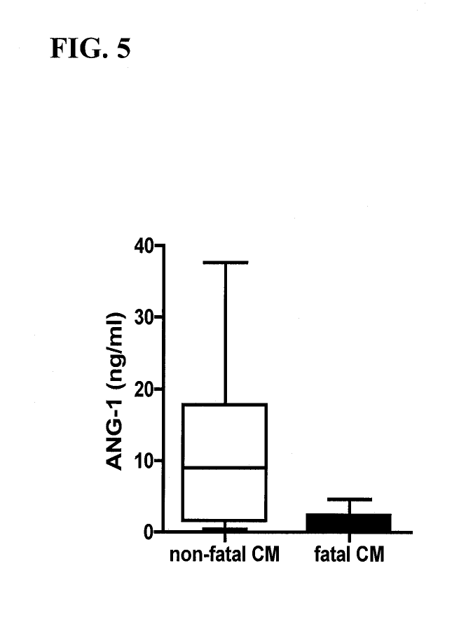

The applicants examined angiopoietin levels at presentation and subsequent survival in children with CM and observed that ANG-1 levels and the ratio of ANG-2:ANG-1 were related to mortality. Higher ANG-1 levels at presentation were associated with protection from fatal CM (median (range): non-fatal CM 9.1 (0.39 to 38) versus fatal CM 0.39 (0.39 to 4.6), p=0.027; FIG. 5) whereas ANG-2:ANG-1 ratios were higher in those who subsequently died of CM (median (range): non-fatal CM 0.13 (0.01 to 82) versus fatal CM 2.6 (1.4 to 13), p=0.013). No patients died in the Thai cohort.

### Example 6

### Dengue Fever and Other Viral Hemorrhagic Fevers

Blood samples for the present study have been collected from individuals with confirmed dengue enrolled in two studies performed by the Walter Reed Thai Dengue Program affiliated with The Armed Forces Research Institute of Medical Sciences (AFRIMS) in Bangkok, Thailand. Patient specimens from two independent study designs are utilized to determine that a dysregulated ANG-2 to ANG-1 ratio is diagnostic and/or prognostic of Dengue Hemorrhagic Fever. A cross sectional study examines cases of classic dengue versus cases of Dengue Shock Syndrome (DSS) and Dengue Hemorrhagic Fever (DHF) and a prospective longitudinal study of confirmed cases of dengue admitted to hospital and enrolled within 3 days of disease onset.

In the cross sectional study, samples taken from consecutive patients with either DF or DHF are analyzed to show that subjects with DHF are identified or differentiated from subjects with DF using angiopoietins as biomarkers. Serial serum samples (collected from all patients at regular intervals) from patients who presented with uncomplicated DF are examined to show that these biomarkers diagnose or predict progression to DSS/DHF.

For all patients, dengue infection was confirmed by serological testing, PCR and viral culture assays.

Concentration of ANG-1 and ANG-2, along with other standard markers of inflammatory disease, such as TNF-alpha, soluble ICAM and other cytokines and chemokines, are tested using standard, commercially available ELISA kits according to the manufacturers' instructions.

Samples are analyzed in a blinded fashion with the diagnosis and clinical information concealed until after all analysis is complete.

### Statistical Analysis

Differences between serum protein levels, sensitivity, specificity, positive predictive value and negative predicted values are analyzed using standard statistical tests using GraphPad Prism (v4.03) or SPSS (v11) software. Receiver operating characteristic (ROC) curves and area under the ROC curves are generated using SPSS (v11). Cutoff values are derived mathematically from the ROC curves, using the point on the ROC curve with the lowest value for the formula: (1−sensitivity)²+(1−specificity)².

### Outcome

Based on the occurrence of vascular leak in DHF and DSS, the balance of ANG-1 and ANG-2, is dysregulated in DHF and DSS compared to DF. More specifically, ANG-1 levels are decreased and ANG-2 levels increased in DHF patients compared to the uncomplicated DF patients. Additionally, the decrease in ANG-1 and increase in ANG-2 (and thus in the ANG-2/ANG-1 ratio) is prognostic of progression to severe disease. In patients who present with Dengue and have mild disease (DF) or progress to severe disease (DHF, DSS), ANG-1 decreases with concurrent ANG-2 increase prior to the development of severe disease.

### Viral Hemorrhagic Fevers and Rickettsial Infections

Similar experiments for viral hemorrhagic fevers such as Marburg and Ebola as well as rickettsial infections are performed as outlined above for Dengue Fever. Based on the experiments disclosed in this application and the occurrence of vascular leak in these infections, the balance of ANG-1 and ANG-2, is dysregulated in severe forms of viral hemorrhagic fevers and rickettsial infections wherein endothelial cell integrity is compromised. More specifically, ANG-1 levels are decreased and ANG-2 levels increased in subjects that with or that develop severe disease compared to the subjects with uncomplicated disease. The decrease in ANG-1 and increase in ANG-2 (and thus in the ANG-2/ANG-1 ratio) is prognostic of progression to severe disease wherein endothelial cell integrity is compromised. In subjects who present with viral hemorrhagic fevers or rickettsial infections, ANG-1 decreases with concurrent ANG-2 increase prior to the development of severe disease.

### Example 7

### Angiopoietins-1 and -2 as Biomarkers for Severe Sepsis

Elevated Angiopoietin-2 levels have been described in patients with severe compared to mild sepsis, and have been suggested to contribute to vascular leak in this disease process (23). Sepsis can be caused by bacterial, viral, fungal, or parasitic infections. Common bacterial causes of sepsis include, but are not limited to Staphylococcus aureus (including MRSA), Streptococcus pyogenes, Streptococcus pneumoniae, Pseudomonas aeruginosa, Klebsiella pneumoniae, etc. In order to investigate the potential role of ANG-1 and ANG-2 levels as biomarkers for disease states wherein endothelial cell integrity is compromised, ANG-1 and ANG-2 protein levels were investigate in serum samples from 43 patients with severe sepsis.

### Study Population

Blood was collected from patients within 24 hours of meeting the definition of severe sepsis. Patients with severe sepsis were identified based on the inclusion criteria described in the PROWESS study (29). The sepsis study cases were all due to definitive/confirmed bacterial or fungal infections (Candida species). Causative bacterial organisms included Staphylococcus aureus, Streptococcus pneumonia, Streptococcus pyogenes, Pseudomonas aeruginosa, Enterococcus faecalis, Klebsiella pneumoniae, Escherichia coli, and others. Serial samples were obtained daily for the first week, and once a week thereafter until study completion. Infection criteria—Patients had to have a known infection or a suspected infection, as evidenced by one or more of the following: white blood cells in a normally sterile body fluid; perforated viscus, radiographic evidence of pneumonia in association with the production of purulent sputum; a syndrome associated with a high risk of infection (e.g., ascending cholangitis). Modified SIRS criteria—Patients had to meet at least three of the following four criteria: a core temperature of ≧38° C. (100.4° F.) or ≦36° C. (96.8° F.); a heart rate of ≧90 beats/min, except in patients with a medical condition known to increase the heart rate or those receiving treatment that would prevent tachycardia; a respiratory rate of ≧20 breaths/min or a PaCO₂ of >32 mm Hg or the use of mechanical ventilation for an acute respiratory process; a white blood cell count of ≧12,000/mm³ or ≦4,000/mm³ or a differential count showing ≧10% immature neutrophils. Criteria for dysfunctional organs or systems—Patients had to meet at least one of the following five criteria: for cardiovascular-system dysfunction, the arterial systolic blood pressure had to be <90 mm Hg or the mean arterial pressure of <70 mm Hg for at least one hour despite adequate fluid resuscitation, adequate intravascular volume status or the use of vasopressors in an attempt to maintain a systolic blood pressure of ≧90 mm Hg or a mean arterial pressure of ≧70 mm Hg; for kidney dysfunction, urine output had to be <0.5 ml/kg of body weight/hr for 1 hour, despite adequate fluid resuscitation; for respiratory-system dysfunction, the ratio of PaO₂ to FiO₂ had to be ≦250 in the presence of other dysfunctional organs or systems or ≦200 if the lung was the only dysfunctional organ; for hematologic dysfunction, the platelet count had to be <80,000/mm³ or to have decreased by 50 percent in the 3 days preceding enrolment; in the case of unexplained metabolic acidosis, the pH had to be ≦7.30 or the base deficit had to be ≧5.0 mmol/liter in association with a plasma lactate level that was >1.5 times the upper limit of the normal value for the reporting laboratory. The exclusion criteria used in this study were: pregnancy or breast-feeding, age <18 years, use of unfractionated heparin to treat an active thrombotic event within 8 hours of blood sampling, and use of low-molecular-weight-heparin at a dose higher than recommended for prophylactic use within 12 hours of blood sampling.

### Testing for ANG-1 and ANG-2 Levels

MODS scores (28) for each patient were recorded on each day (i.e. days 1-7; 14; 21; and 29 if available) that patient serum samples were obtained. ANG-1 and ANG-2 levels were measured in duplicate for each patient sample using an ELISA based assay. The collected data was analyzed using Excel, Prism Graphpad and SPSS.

### Results

Angiopoietin-2 levels were observed to correlate with MODS scores on the same day (p<0.0001). However no correlation was observed between MODS scores and angiopoietin-1 levels on the same day (p=0.976).

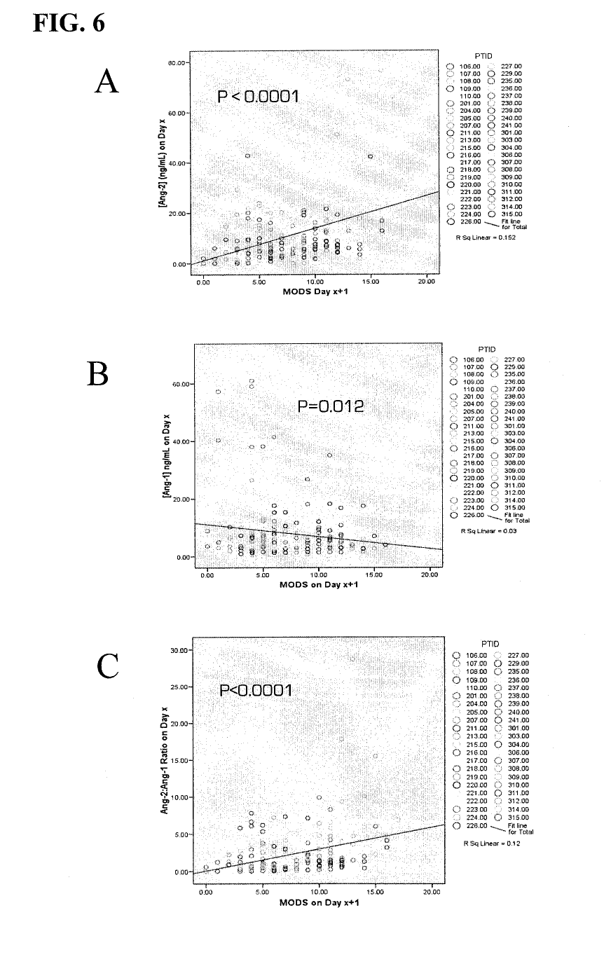

To investigate the role of angiopoietin biomarkers for identifying patients at risk of developing severe sepsis, or developing a more severe form of disease as indicated by higher MODS scores, ANG-1 and ANG-2 serum levels on day X were correlated with MODS scores on day X+1 (i.e. the next day). As shown in FIG. 6A, angiopoietin-2 levels were found to be significantly correlated with next-day MODS scores (p<0.0001). Furthermore, angiopoietin-1 levels were also significantly correlated with next day MODS scores (p=0.012) (FIG. 6B) and the ANG-2:ANG-1 ratio was also significantly correlated with next day MODS scores (p<0.0001) (FIG. 6C).

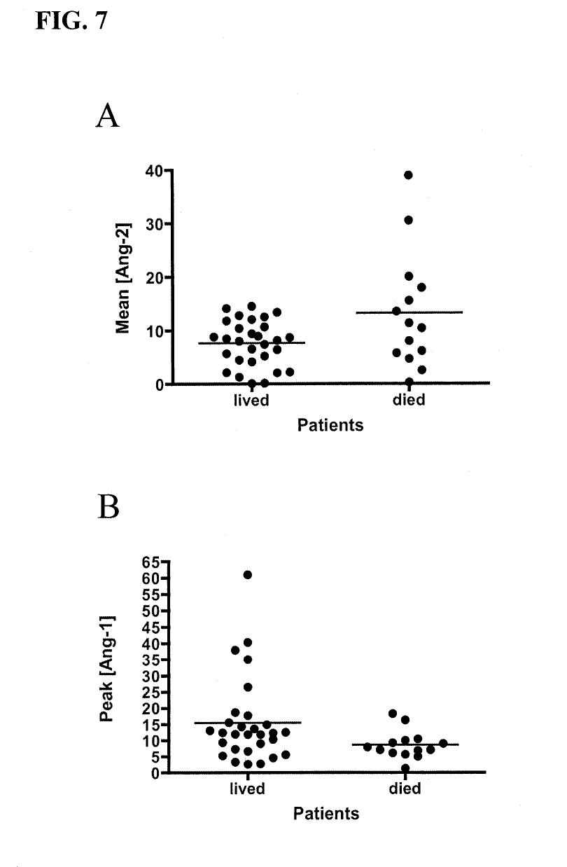

ANG-2 and ANG-1 levels were also examined as predictors of mortality in the study population. As shown in FIG. 7A, mean ANG-2 levels did not significantly distinguish between patients that lived or died. However, as shown in FIG. 7B peak ANG-1 levels were significant biomarkers for distinguishing between patients that lived and died (p=0.027).

While the present invention has been described with reference to particular embodiments and examples, the invention is not limited to the disclosed embodiments and examples. The invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Snow, R. W., C. A. Guerra, A. M. Noor, H. Y. Myint, and S. I. Hay. 2005. The global distribution of clinical episodes of Plasmodium falciparum malaria. Nature 434:214-217.
2. 1990. Severe and complicated malaria. World Health Organization, Division of Control of Tropical Diseases. Trans R Soc Trop Med Hyg 84 Suppl 2:1-65.
3. Newton, C. R., and S. Krishna. 1998. Severe falciparum malaria in children: current understanding of pathophysiology and supportive treatment. Pharmacol Ther 79:1-53.
4. Kain, K. C., M. A. Harrington, S. Tennyson, and J. S. Keystone. 1998. Imported malaria: prospective analysis of problems in diagnosis and management. Clin Infect Dis 27:142-149.
5. Taylor, T. E., Fu W. J., Carr, R. A., et al. Differentiating the pathologies of cerebral malaria by postmortem parasite counts. Nat Med 2004 February; 10(2):143-5.
6. Marsh, K., D. Forster, C. Waruiru, I. Mwangi, M. Winstanley, V. Marsh, C. Newton, P. Winstanley, P. Warn, N. Peshu, and et al. 1995. Indicators of life-threatening malaria in African children. N Engl J Med 332:1399-1404.
7. Jaffar, S., M. B. Van Hensbroek, A. Palmer, G. Schneider, and B. Greenwood. 1997. Predictors of a fatal outcome following childhood cerebral malaria. Am J Trop Med Hyg 57:20-24.
8. Dzeing-Ella, A., P. C. Nze Obiang, R. Tchoua, T. Planche, B. Mboza, M. Mbounja, U. Muller-Roemer, J. Jarvis, E. Kendjo, E. Ngou-Milama, P. G. Kremsner, S. Krishna, and M. Kombila. 2005. Severe falciparum malaria in Gabonese children: clinical and laboratory features. Malar J 4:1.
9. Allen, S. J., A. O'Donnell, N. D. Alexander, and J. B. Clegg. 1996. Severe malaria in children in Papua New Guinea. Qjm 89:779-788.
10. Akanmori, B. D., J. A. Kurtzhals, B. Q. Goka, V. Adabayeri, M. F. Ofori, F. K. Nkrumah, C. Behr, and L. Hviid. 2000. Distinct patterns of cytokine regulation in discrete clinical forms of Plasmodium falciparum malaria. Eur Cytokine Netw 11:113-118.
11. Grau, G. E., T. E. Taylor, M. E. Molyneux, J. J. Wirima, P. Vassalli, M. Hommel, and P. H. Lambert. 1989. Tumor necrosis factor and disease severity in children with falciparum malaria. N Engl J Med 320:1586-1591.
12. Kern, P., C. J. Hemmer, J. Van Damme, H. J. Gruss, and M. Dietrich. 1989. Elevated tumor necrosis factor alpha and interleukin-6 serum levels as markers for complicated Plasmodium falciparum malaria. Am J Med 87:139-143.
13. Kwiatkowski, D., A. V. Hill, I. Sambou, P. Twumasi, J. Castracane, K. R. Manogue, A. Cerami, D. R. Brewster, and B. M. Greenwood. 1990. TNF concentration in fatal cerebral, non-fatal cerebral, and uncomplicated Plasmodium falciparum malaria. Lancet 336:1201-1204.
14. Lyke, K. E., R. Burges, Y. Cissoko, L. Sangare, M. Dao, I. Diarra, A. Kone, R. Harley, C. V. Plowe, O. K. Doumbo, and M. B. Sztein. 2004. Serum levels of the proinflammatory cytokines interleukin-1 beta (IL-1beta), IL-6, IL-8, IL-10, tumor necrosis factor alpha, and IL-12(p 70) in Malian children with severe Plasmodium falciparum malaria and matched uncomplicated malaria or healthy controls. Infect Immun 72:5630-5637.
15. Tchinda, V. H., A. D. Tadem, E. A. Tako, G. Tene, J. Fogako, P. Nyonglema, G. Sama, A. Zhou, and R. G. Leke. 2007. Severe malaria in Cameroonian children: correlation between plasma levels of three soluble inducible adhesion molecules and TNF-alpha. Acta Trop 102:20-28.
16. Gimenez, F., S. Barraud de Lagerie, C. Fernandez, P. Pino, and D. Mazier. 2003. Tumor necrosis factor alpha in the pathogenesis of cerebral malaria. Cell Mol Life Sci 60:1623-1635.
17. Turner, G. D., H. Morrison, M. Jones, T. M. Davis, S. Looareesuwan, I. D. Buley, K. C. Gatter, C. I. Newbold, S. Pukritayakamee, B. Nagachinta, and et al. 1994. An immunohistochemical study of the pathology of fatal malaria. Evidence for widespread endothelial activation and a potential role for intercellular adhesion molecule-1 in cerebral sequestration. Am J Pathol 145:1057-1069.
18. Jakobsen, P. H., S. Morris-Jones, A. Ronn, L. Hviid, T. G. Theander, I. M. Elhassan, I. C. Bygbjerg, and B. M. Greenwood. 1994. Increased plasma concentrations of sICAM-1, sVCAM-1 and sELAM-1 in patients with Plasmodium falciparum or P. vivax malaria and association with disease severity. Immunology 83:665-669.
19. Turner, G. D., V. C. Ly, T. H. Nguyen, T. H. Tran, H. P. Nguyen, D. Bethell, S. Wyllie, K. Louwrier, S. B. Fox, K. C. Gatter, N. P. Day, T. H. Tran, N. J. White, and A. R. Berendt. 1998. Systemic endothelial activation occurs in both mild and severe malaria. Correlating dermal microvascular endothelial cell phenotype and soluble cell adhesion molecules with disease severity. Am J Pathol 152:1477-1487.
20. Hollestelle, M. J., C. Donkor, E. A. Mantey, S. J. Chakravorty, A. Craig, A. O. Akoto, J. O'Donnell, J. A. van Mourik, and J. Bunn. 2006. von Willebrand factor propeptide in malaria: evidence of acute endothelial cell activation. Br J Haematol 133:562-569.
21. Fiedler, U., and H. G. Augustin. 2006. Angiopoietins: a link between angiogenesis and inflammation. Trends Immunol 27:552-558.
22. Loughna, S., and T. N. Sato. 2001. Angiopoietin and Tie signaling pathways in vascular development. Matrix Biol 20:319-325.
23. Parikh, S. M., T. Mammoto, A. Schultz, H. T. Yuan, D. Christiani, S. A. Karumanchi, and V. P. Sukhatme. 2006. Excess circulating angiopoietin-2 may contribute to pulmonary vascular leak in sepsis in humans. PLoS Med 3:e46.
24. Altman, D. G., and J. M. Bland. 1994. Diagnostic tests 3: receiver operating characteristic plots. Bmj 309:188.
25. Gubler, D. J. 1998. Dengue and dengue hemorrhagic fever. Clin Microbiol Rev 11:480-496.
26. Marty, A. M., P. B. Jahrling, and T. W. Geisbert. 2006. Viral hemorrhagic fevers. Clin Lab Med 26:345-386, viii.
27. John C C, Opika-Opoka R, Byarugaba J, Idro R, Boivin M J. Low levels of RANTES are associated with mortality in children with cerebral malaria. J Infect Dis. 2006 Sep. 15; 194(6):837-45.
28. Marshall J C, Cook, D J, Chistou N V, Bernard G R, Sprung C L, Sibbald W J. Multiple organ dysfunction score: a reliable descriptor of a complex clinical outcome. Crit Care Med 1995; 23:1638-1652
29. Bernard G R, Vincent J L, Laterre P F et al. Efficacy and safety of recombinant human activated protein C for severe sepsis. N Engl J Med 2001; 344:699-709.
30. Aird W C. The role of the endothelium in severe sepsis and multiple organ dysfunction syndrome. Blood. 2003 May 15; 101(10):3765-77.
31. Vallet B. Bench-to-bedside review: endothelial cell dysfunction in severe sepsis: a role in organ dysfunction? Crit. Care. 2003 April; 7(2):130-8.
32. Lemieux C, Maliba R, Favier J, Théorêt J F, Merhi Y, Sirois M G. Angiopoietins can directly activate endothelial cells and neutrophils to promote proinflammatory responses. Blood. 2005 Feb. 15; 105(4):1523-30.

1. A method of identifying a subject having, or at risk of developing, an infectious disease state wherein endothelial cell integrity is compromised comprising:
(a) determining a test ANG-1 level in a sample from a subject; and
(b) comparing the test ANG-1 level to a control level wherein a lower test ANG-1 level compared to the control level is indicative of the subject having or developing said infectious disease state.

2. The method of claim 1, further comprising determining a test ANG-2 level in a subject, and comparing the test ANG-2 level to a control level, wherein increased test ANG-2 level is indicative of the subject having or developing said infectious disease state.

3. The method of claim 2, further comprising determining the ratio of test ANG-2 level to test ANG-1 level, where an increase in the ratio of test ANG-2/test ANG-1 compared to a control ratio or a decrease in the ratio of test ANG-1/test ANG-2 compared to a control ratio is indicative of the subject having or developing said infectious disease state.

4. The method of claim 1, wherein the infectious disease state comprises severe malaria or cerebral malaria.

5. The method of claim 4 wherein the control level of ANG-1 is between 15 and 22 ng/ml.

6. The method of claim 1, wherein the infectious disease state comprises sepsis, severe sepsis or septic shock.

7. The method of claim 6, wherein severe sepsis is caused by a bacterial, viral, fungal, or parasitic infection.

8. The method of claim 1 wherein the control level is derived from a Receiver Operating Characteristic (ROC) curve.

9. The method of claim 1, wherein the infectious disease state comprises Dengue Hemorrhagic Fever or Dengue Shock Syndrome.

10. The method of claim 1, wherein the infectious disease state comprises a viral hemorrhagic fever.

11. The method of claim 10 wherein the viral hemorrhagic fever is caused by the Marburg virus, Ebola virus or a rickettsial infectious agent.

12. The method of claim 1, wherein the identity of the infectious disease is not known.

13. The method of claim 1, wherein the test sample comprises serum.

14. The method of claim 1, wherein the subject is a human.

15. The method of claim 1, wherein the control level is determined from a test sample from said subject at an earlier time point.

16. The method of claim 15, wherein said infectious disease state is selected from the group consisting of severe malaria, cerebral malaria, sepsis, severe sepsis and septic shock.

17. A kit for determining whether a subject has, or is at risk for developing an infectious disease state wherein endothelial cell integrity is compromised, comprising an antibody directed against ANG-1 and/or an antibody directed against ANG-2.

18. The kit of claim 17, wherein the antibodies are detectably labeled.

19. The kit of claims 17 or 18, further comprising a medium suitable for formation of an antigen-antibody complex, and reagents for detection of the antigen-antibody complexes and instructions for the use thereof.

20. The method of claim 1, wherein the infectious disease state comprises an infectious disease state caused by exposure to a biowarfare agent.

21. (canceled)
